(19) United States
(12) Patent Application Publication
(10) Pub. No.: US 2005/0159619 A1
(43) Pub. Date: Jul. 21, 2005

(54) Catalyst for production of unsaturated aldehyde and unsaturated carboxylic acid and process for producing the same

(75) Inventors: Masahide Kondo, Hiroshima (JP); Seiichi Kawato, Hiroshima (JP); Toru Kuroda, Hiroshima (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Mitsubishi Rayon Co., Ltd., Tokyo (JP)

(21) Appl. No.: 10/504,143

(22) PCT Filed: Feb. 19, 2003
(86) PCT No.: PCT/JP03/01769

(30) Foreign Application Priority Data
Feb. 19, 2002 (JP) 2002-42215

(51) Int. Cl. B01J 23/31; C07C 51/16; C07C 45/35

(52) U.S. Cl. 562/546; 502/311; 568/479

(57) ABSTRACT

Prepared is a catalyst for use in the production of an unsaturated aldehyde and an unsaturated carboxylic acid in which catalytic activity is high and selectivity is excellent by a preparation process containing the steps of preparing catalyst component particles as dried particles prepared by spray drying an aqueous slurry containing molybdenum, bismuth and iron or as calcined particles prepared by further heat treating the dried particles; mixing and kneading the catalyst component particles with at least a liquid; first-molding the kneaded material; and second-molding the first-molded product to a finished shape using a piston molding machine.

### TECHNICAL FIELD

The present invention relates to a catalyst for use in the preparation of an unsaturated aldehyde and an unsaturated carboxylic acid, the preparation process thereof and the preparation process of the unsaturated aldehyde and unsaturated carboxylic acid that uses such catalyst, wherein the catalyst comprises at least molybdenum, bismuth and iron, and is used for preparation of an unsaturated aldehyde and an unsaturated carboxylic acid by vapor-phase catalytic oxidation using molecular oxygen from propylene, isobutylene, tert-butyl alcohol (hereinafter referred to TBA) or methyl tert-butyl ether (hereinafter referred to MTBE).

### BACKGROUND ART

Heretofore, a large number of proposals have been made concerning catalysts and the preparation process for such catalysts, that can be used for the preparation of an unsaturated aldehyde and an unsaturated carboxylic acid from propylene, isobutylene, TBA or MTBE by vapor-phase catalytic oxidation. Many of these catalysts have components containing at least molybdenum, bismuth and iron, wherein molded catalysts having such a composition are being used for industrial purposes. These catalysts are classified into extrusion-molded catalysts, carrier-supported catalysts and so on, depending on their molding method. Usually, an extrusion-molded catalyst is prepared by kneading particles comprising the catalyst components then carrying out extrusion-molding. On the other hand, a carrier-supported catalyst is prepared by supporting a powder comprising the catalyst components on a carrier.

With respect to extrusion-molded catalysts, various techniques have been proposed; which include, for example, a process for improving strength and selectivity by adding graphite or an inorganic fiber during preparation (Japanese Patent Laid-Open No. 60-150834); and a process which adds a certain type of cellulose derivative during extrusion-molding of the catalyst (Japanese Patent Laid-Open No. 7-16464). In addition, Japanese Patent Laid-Open No. 2000-70719 discloses extrusion-molding by kneading, with (Examples) or without (Comparative Examples) addition of a surfactant, the calcined particles of dried particles obtained using a spray dryer. These are all preparation processes using one-step molding.

Japanese Patent Laid-Open No. 2000-71313 discloses a molding process of a porous molded product, in which material charged into a piston type extruder can be molded into a shape that is easily charged into the piston extruder cylinder by using a screw extruder or the like beforehand. While Example 4 of that document specifically discloses a molding process for an isobutylene oxidation catalyst comprising molybdenum, bismuth and iron as one example of this, the material charged into the piston type extruder had not been pre-molded.

However, the oxidation catalysts obtained by these commonly-known processes are still insufficient as industrial catalysts in terms of their catalytic activity and desired product selectivity.

### DISCLOSURE OF THE INVENTION

The present invention was carried out in order to resolve the above-described problems. It is an object thereof to provide a catalyst for use in the preparation of an unsaturated aldehyde and unsaturated carboxylic acid which is excellent in catalytic activity and in selectivity of unsaturated aldehyde and unsaturated carboxylic acid, a preparation process of such catalyst, and a process for producing an unsaturated aldehyde and unsaturated carboxylic acid at a high yield using this catalyst.

The preparation process of a catalyst for an unsaturated aldehyde and an unsaturated carboxylic acid according to the present invention is a process for preparing an extrusion-molded catalyst containing at least molybdenum, bismuth and iron for use in the production of an unsaturated aldehyde and an unsaturated carboxylic acid by vapor-phase catalytic oxidation of at least one of propylene, isobutylene, tert-butyl alcohol or methyl tert-butyl ether using molecular oxygen, said process comprising: a step of preparing catalyst component particles as dried particles prepared by spray drying an aqueous slurry comprising molybdenum, bismuth and iron or as calcined particles prepared by further heat treating said dried particles; a step of mixing and kneading said catalyst component particles with at least a liquid; a first-molding step of molding the kneaded material; and a second-molding step of molding the first-molded product to a finished shape using a piston molding machine.

In this preparation process, the shape of the first-molded product molded by the above-described first-molding step is cylindrical, and preferably has a diameter of 0.5 times or larger and less than 1 times of the cylinder diameter of the piston molding machine used in the second-molding step.

The specific gravity of said first-molded product is preferably from 1.1 to 2.7 kg/L.

In addition, the average particle diameter of the catalyst component particles is from 10 to 150 m. The average particle compression strength of the catalyst component particles is preferably from 9.8×10⁻⁴ to 9.8×10⁻² N. The bulk specific gravity of the catalyst component particles is preferably from 0.5 to 1.8 kg/L.

Vacuum deaeration is preferably not carried out during molding of the first-molded product into the finished shape using the piston molding machine of the second-molding. During first-molding, a screw extruder is preferably used for molding.

The amount of the liquid to be mixed with the catalyst component particles is preferably from 35 to 55 parts by mass per 100 parts by mass of the catalyst component particles.

The catalyst component particles are preferably calcined particles.

The present invention also relates to the catalyst for preparing the unsaturated aldehyde and unsaturated carboxylic acid according to the present invention prepared in accordance with the above-described preparation processes. The catalyst shape is in particular a ring shape, and preferably has an outer diameter of from 3 to 15 mm.

The present invention further relates to a preparation process of an unsaturated aldehyde and an unsaturated carboxylic acid, which uses the above-described catalyst in vapor-phase catalytic oxidation of at least one of propylene, isobutylene, TBA or MTBE using molecular oxygen.

### BEST MODE FOR CARRYING OUT THE INVENTION

The catalyst for preparing an unsaturated aldehyde and an unsaturated carboxylic acid according to the present invention is an extrusion-molded catalyst prepared using a below-described preparation process, which is used in preparing an unsaturated aldehyde and unsaturated carboxylic acid by subjecting the reaction raw material, i.e. propylene, isobutylene, TBA or MTBE, to vapor-phase catalytic oxidation using molecular oxygen.

The above-described catalyst is a catalyst comprising at least molybdenum, bismuth and iron as a catalyst component. The catalyst may also comprise catalyst components other than molybdenum, bismuth and iron, such as silicon, cobalt, nickel, chromium, lead, manganese, calcium, magnesium, niobium, silver, barium, tin, tantalum, zinc, phosphorus, boron, sulfur, selenium, tellurium, cerium, tungsten, antimony, titanium, lithium, sodium, potassium, rubidium, cesium and thallium.

For example, the catalyst preferably has a composition represented by the following general formula (I).
MoₐBi_{b}Fe_{c}M_{d}XₑY_{f}Z_{g}SiₕOᵢ (I)
wherein Mo, Bi, Fe, Si and O independently represent molybdenum, bismuth, iron, silicon and oxygen, respectively; M represents at least one element selected from the group consisting of cobalt and nickel; X represents at least one element selected from the group consisting of chromium, lead, manganese, calcium, magnesium, niobium, silver, barium, tin, tantalum and zinc; Y represents at least one element selected from the group consisting of phosphorus, boron, sulfur, selenium, tellurium, cerium, tungsten, antimony and titanium; and Z represents at least one element selected from the group consisting of lithium, sodium, potassium, rubidium, cesium and thallium. Moreover, a, b, c, d, e, f, g, h and i represent the atomic ratios of the aforesaid elements. When a=12, they may be chosen so that b=0.01 to 3, c=0.01 to 5, d=1 to 12, e=0 to 8, f=0 to 5, g=0.001 to 2 and h=0 to 20; i is the atomic ratio of oxygen which satisfies the valence of each constituent element.

Preparation of the catalyst for preparing an unsaturated aldehyde and an unsaturated carboxylic acid according to the present invention comprises: (1) a step of preparing catalyst component particles; (2) a step of kneading the obtained catalyst component particles; (3) a step of first-molding the obtained kneaded material; (4) a step of second-molding the obtained first-molded material using a piston molding machine; and usually further comprises (5) a step of drying and/or heat-treating the molded product.

In step (1) for preparing the catalyst component particles, dried particles are prepared by spray drying an aqueous slurry comprising molybdenum, bismuth and iron. Spray drying has the characteristic that the shape of the obtained particles is a uniform spherical shape.

No particular restriction is placed on the method for preparing the aqueous slurry, provided that it does not cause a significant localization of the components. Any of the various techniques which have heretofore been well-known can be used, such as the precipitation method and the oxide mixing method. Compounds such as oxides, sulfates, nitrates, carbonates, hydroxides, ammonium salts and halides containing the element of the catalyst component can be used as the raw material for the catalyst component. For example, the raw material when molybdenum is a catalyst component includes ammonium paramolybdate, molybdenum trioxide and the like. Furthermore, either one type per respective element or two or more can be employed as the raw material for the catalyst component.

Spray drying can be carried out using a spray dryer equipped with, for example, a rotating disk centrifugal atomizer, a two-fluid nozzle atomizer and the like. The spray drying conditions, such as inlet temperature and outlet temperature, may be suitably set so as to obtain a desired average particle diameter. For example, the standard drying conditions when spray drying an aqueous slurry containing 35 to 55 mass % of solid matter using a spray dryer equipped with a rotating disk centrifugal atomizer are an inlet temperature of from 100 to 500° C., an outlet temperature from 100 to 200° C. and an atomizer revolution speed of from 8000 to 20000 rpm.

The dried particles obtained in this way sometimes contain salts, such as nitrates, originating from the catalyst raw material and the like. If a molded product molded from dried particles containing a large amount of salts is calcined to decompose the salts, the strength of the molded product may be reduced. For this reason, the particles are not only dried, but are also preferably calcined at this point to give calcined particles. No particular restrictions are placed on the calcining conditions, but usually the particles are calcined at a temperature in the range of 200 to 600° C. in the presence of, or under the flow of, oxygen, air or nitrogen. The calcining time is suitably chosen according to the catalyst raw material, the desired catalyst and the like.

Hereinafter, the dried particles and the calcined particles comprising the catalyst component shall be together referred to as catalyst component particles.

When molding is carried out without crushing the catalyst component particles, if the average particle diameter increases, large voids, i.e. large pores is formed between the particles after molding, whereby selectivity tends to improve. On the other hand, if the average particle diameter decreases, the number of contact points between particles per unit volume increases, whereby the mechanical strength of the obtained molded catalyst tends to improve. In consideration of these points, the average particle diameter is preferably 10 μm or greater, while 150 μm or less is preferable. An average particle diameter in the range of from 10 μm to 150 μm has an excellent balance between selectivity and mechanical strength. Further, an average particle diameter of 20 μm or more is more preferable, 45 μm or more being especially preferable, while 100 μm or less is more preferable and 65 μm or less being especially preferable.

A greater bulk specific gravity tends to withstand molding, while a lower bulk specific gravity tends to have higher activity and selectivity. Therefore, in view of handling property during molding and catalytic performance, a bulk specific gravity in the range of from 0.5 to 1.8 kg/L is preferable. In this range sufficient strength able to withstand molding can be obtained, so that the particles are not easily crushed during molding, while the activity and selectivity of the catalyst are also high. From 0.8 to 1.2 kg/L is particularly preferable. Here, bulk specific gravity is the value measured using a method in accordance with JIS K6721. The bulk specific gravity of the catalyst component particles can be adjusted, for example, by adjusting the concentration of the aqueous slurry to be spray dried, the mixing rate or stirring rate during preparation of the aqueous slurry, the concentration of the slurry and the like.

Greater average particle compression strength of the catalyst component particles tends to withstand molding, while lower average particle compression strength tends to have higher activity and selectivity. Therefore, in view of handling property during molding and catalytic performance, an average particle compression strength in the range of 9.8×10⁻⁴ to 9.8×10⁻² N is preferable, and from 4.9×10⁻³ to 4.9×10⁻² N is particularly preferable. The average particle compression strength of the catalyst component particles can be adjusted, for example, by adjusting the concentration of the aqueous slurry to be spray dried, the mixing rate or stirring rate during preparation of the aqueous slurry, the concentration of the slurry and the like.

Next, in step (2) for kneading the obtained catalyst component particles (i.e. dried particles or calcined particles), a mixture of at least the catalyst component particles and a liquid are kneaded to give a kneaded material.

Examples of a preferable liquid to be used in this step include water and alcohols. Examples of the alcohol include lower alcohols, such as ethanol, methyl alcohol, propyl alcohol and butyl alcohol. While these liquids can be used alone or in combination of two or more, it is preferable to use water because it is economic and easy to handle.

The amount of liquid used may be suitably chosen according to the type and size of the catalyst component particles, the type of liquid and the like. However, usually the amount is in the range of from 10 to 70 parts by mass per 100 parts by mass of the catalyst component particles. Since a larger amount of liquid used allows for smoother extrusion-molding, the spherical particles are less easily crushed, whereby large voids, i.e. large pores, form in the dried and calcined molded product, which tends to improve selectivity. Therefore, the used amount of liquid is preferably 20 parts by mass or more, more preferably 30 parts by mass or more, and especially preferably 35 parts by mass or more per 100 parts by mass of the catalyst component particles. On the other hand, a smaller amount of liquid used reduces adhesion during molding, which improves handling property. Further, the smaller the used amount of liquid, the denser the molded product becomes, which tends to improve the strength of the molded product. Therefore, the used amount of liquid is preferably 60 parts by mass or less, more preferably 50 parts by mass or less, and even more preferably 45 parts by mass or less per 100 parts by mass of the catalyst component particles.

In step (2), it is preferable to add a molding aid (molding assisting material) such as an organic binder to the mixture containing catalyst component particles and a liquid, because strength improves. Examples of such molding aids include methylcellulose, ethylcellulose, carboxymethylcellulose, carboxymethylcellulose sodium salt, hydroxyethyl cellulose, hydroxypropyl cellulose, hydroxypropyl methylcellulose, hydroxyethyl methylcellulose, hydroxybutyl methylcellulose, ethylhydroxyethyl cellulose and hydroxypropyl cellulose. The added amount of these molding aids is preferably 0.1 parts by mass or more per 100 parts by mass of the particles comprising the catalyst component, and 2 parts by mass or more is especially preferable. From the point that post-treatment, such as heat-treatment after molding, becomes easier, the added amount of the molding aid is preferably 10 parts by mass or less per 100 parts by mass of the particles comprising the catalyst component, and 6 parts by mass or less is especially preferable.

A conventionally known additive can also be added to the above mixture, examples thereof including inorganic compounds such as graphite and diatomaceous earth; and inorganic fibers such as glass fiber, ceramic fiber and carbon fiber.

No particular restriction is placed on the apparatus used for kneading the mixture containing catalyst component particles and a liquid. For example, there may be used a batch type kneader having double-arm agitating blades, and continuous type kneaders such as an axial-rotation reciprocating type and a self-cleaning type kneader. However, a batch type kneader is preferred because it has the advantage of being able to carry out kneading while the state of the kneaded material is monitored. The kneading completion point is usually judged by time, visual observation or touch feeling.

Next, in the first-molding step (3), the kneaded material obtained in the kneading step is molded into a first-molded product using an apparatus such as an extruder or a press machine. An apparatus can also be employed which performs the kneading and the first-molding in succession (one-pass). Here, it is preferable to carry out the kneading using a batch type kneader and the first-molding using a screw extruder, in terms of the fact that kneading can be carried out while the kneading state is monitored and in terms of productivity.

The first-molding shape is not particularly restricted, although it is preferably a cylindrical shape having a diameter of 0.5 or more times and less than 1 times of the diameter of the piston molding machine carrying out the second-molding. The smaller the diameter of the cylindrical first-molded product, the easier it is to charge the first-molded product into the piston molding machine. However, when the diameter is from 0.5 or more times up to less than 1 times, the greater the diameter is, the harder it is for excess air to enter during second-molding, whereby the load on the catalyst particles is lessened. In addition, the volume inside the cylinder can be used effectively. Thus, there is the advantage that productivity is improved when producing the same amount of molded product because the number of first-molding and second-molding cycles can be reduced. Further, in this range, the greater the diameter of the first-molding, the lower the mechanical load placed on the catalyst particles, which is advantageous in terms of pore control. Therefore, in particular, a cylindrical shape having a diameter of from 0.8 or more times to less than 1 times of the cylinder diameter of the piston molding machine is preferable.

In addition, the greater the specific gravity of the prepared first-molded product, the greater the strength of the finished catalyst, while the smaller the specific gravity, the more the selectivity of the finished catalyst improves. Therefore, in consideration of the strength and selectivity of the finished catalyst, the specific gravity of the first-molded product is preferably in the range of from 1.1 to 2.7 kg/L, more preferably in the range from of 1.5 to 2.3 kg/L, and still more preferably in the range of from 1.7 to 2.1 kg/L. Here, specific gravity means the value calculated by dividing the weight of the first-molded product comprising the liquid used in kneading by the volume of the first-molded product.

Next, in the second-molding step (4), the obtained first-molding is molded into a finished shape using a piston molding machine.

Piston molding is effective to reduce bending during extrusion and the like, which improves the product yield. Furthermore, molding can be carried out with a uniform strength, whereby the intermixing of excess air is small. This allows a uniform molded product to be achieved, in which the powder ratio in charging the finished catalyst into the reaction tube is reduced and selectivity is improved.

Compared with the case where a first-molding is not carried out where an indeterminate shaped kneaded material is directly (using a piston extruder and the like) extrusion-molded into a finished shape, the present invention allows the smoother extrusion-molding because the shape is already formed after the first-molding. Thus, excess load is not placed on the catalyst particles during molding, a soft molding can be achieved that does not destroy the catalyst particles, and preferable pores can be manifested in the finished catalyst. These attain a catalyst with excellent in catalytic activity and in selectivity of unsaturated aldehyde and unsaturated carboxylic acid.

In the second-molding step, it is preferable to not carry out vacuum deaeration, so that the pore volume of the catalyst is not reduced.

The shape of the molded catalyst obtained from extrusion-molding from the second-molding is not particularly restricted, and may be molded into an arbitrary shape such as a ring shape, a cylindrical shape and stellate pillars. Here, while the shape of the molded catalyst is not particularly restricted, because a soft molding can be achieved compared with conventional mold processes in which extrusion-molding into the finished shape is carried out in a single stage, or indeterminate shaped particles are piston molded, the present process is preferable for a ring shape, which places a comparatively large load on the catalyst particles during molding, in particular for a ring shape having an outer diameter of from 3 to 15 mm. Further, ring shapes are also referred to using a different term, “hollow cylindrical shape.”

Next, in the drying and/or calcining step (5) of the molded catalyst, the obtained molded catalyst is dried and/or calcinated to give the catalyst (product).

No particular restriction is placed on the drying method employed in this step, wherein a well-known drying method such as hot-air drying, humidity drying, far-infrared drying and microwave drying can be arbitrarily employed. The drying conditions may be appropriately chosen so that the desired water content can be achieved.

Usually, the dried molded product is subjected to calcining. However, where in step (1) the particles were calcined, and an organic binder and the like was not used, calcining of the molded catalyst can be omitted. Accordingly, the dried molded product is calcined as required. The calcining conditions are not particularly restricted, and commonly known calcining conditions can be employed. Usually, calcining is carried out in a temperature range of from 200 to 600° C., in the presence of, or under the flow of oxygen, air or nitrogen. The calcining time is suitably chosen according to the desired catalyst.

The catalyst obtained in this way is a uniform molded product, because uniform catalyst component particles are molded using a uniform strength. Furthermore, as the finished catalyst, if a uniform molded product is charged into the reaction tube used during preparation of the below-described unsaturated aldehyde and unsaturated carboxylic acid, the powder ratio can be reduced because there are no molded products that have an extremely small strength.

The powder ratio is defined as provided below. Molded catalyst (1000 g) was charged into a stainless steel cylinder container arranged perpendicularly to the horizontal direction having an inner diameter of 2.75 cm and a length of 6 m by dropping catalyst from the top of the container. The molded catalyst was subsequently recovered from a lower portion of the container. From the recovered molded catalyst, if the amount that did not pass through a sieve having apertures of 1.19 mm is taken as X g:
Powder ratio (%)=((1000−X)/1000)×100
If the powder ratio decreases, a catalyst having high selectivity can be achieved, since the pressure loss is small. In addition, compared with the case of production from catalyst component particles obtained by a method other than spray drying, excess load is not placed on the catalyst particles during molding because extrusion-molding can be carried out more smoothly, whereby a soft molding can be achieved that does not destroy the catalyst particles. Since preferable pores can be achieved in the finished catalyst, a catalyst can be obtained which is excellent in catalytic activity and unsaturated aldehyde and unsaturated carboxylic acid selectivity.

Next, a preparation process of an unsaturated aldehyde and unsaturated carboxylic acid will be explained.

The catalyst according to the present invention is charged into a reaction tube made of, for example, stainless steel, to form a catalyst layer. The feeding gas containing the reaction raw material, i.e. propylene, isobutylene, TBA or MTBE, and molecular oxygen were supplied to this catalyst layer, whereby the reaction raw material was subjected to vapor-phase catalytic oxidation.

The reaction raw material, i.e. propylene, isobutylene, TBA or MTBE, can be used singly, or in combination of 2 kinds or more. While the concentration of these reaction raw materials in the feeding gas may vary over a wide range, from 1 to 20 volume % is suitable and from 3 to 10 volume % is preferable.

While it is economical to use air as the molecular oxygen, if necessary air enriched with pure oxygen may be used. The oxygen concentration in the feeding gas is defined by the molar ratio in relation to the reaction raw materials. This value is preferably from 0.3 to 4 molar ratio to 1 mole of the total raw material, and in particular from 0.5 to 3 molar ratio is preferable.

The feeding gas preferably comprises water in addition to the reaction raw material and molecular oxygen, in which the water concentration in the feeding gas is preferably from 1 to 45 volume %. It is also preferable that the feeding gas be diluted with an inert gas.

The reaction pressure is preferably from atmospheric pressure to several hundred kPa. The reaction temperature can be chosen in the range of from 200 to 450° C., although the range of from 250 to 400° C. is especially preferable. The contact time is preferably 1.5 to 15 seconds.

In the reaction tube, the catalyst may be diluted with an inert carrier such as silica, alumina, silica-alumina, silicon carbide, titania, magnesia, ceramic balls or stainless steel.

Preparation examples from the catalyst according to the present invention include preparation of acrolein and acrylic acid from the oxidation of propylene, and preparation of methacrolein and methacrylic acid from the oxidation of isobutylene, TBA or MTBE.

### EXAMPLES

The present invention will now be more specifically explained with reference to the following examples and comparative examples.

In the description of the examples and the comparative examples, the term “parts” refers to parts by mass. For kneading, a batch type kneader equipped with double-arm agitating blades was used. Analysis of the raw material gas and the reaction raw material was carried out by gas chromatography.

In the examples and the comparative examples, the degree of conversion of the raw material (olefin, TBA or MTBE) (hereinafter referred to as the ratio of conversion), and the selectivity for the unsaturated aldehyde or unsaturated carboxylic acid formed were calculated according to the following equations.
Ratio of conversion (%)=A/B×100
Unsaturated aldehyde selectivity (%)=C/A×100
Unsaturated carboxylic acid selectivity (%)=D/A×100
where A is the number of moles of the reacted raw material olefin, TBA or MTBE; B is the number of moles of the supplied raw material olefin, TBA or MTBE; C is the number of moles of the formed unsaturated aldehyde; and D is the number of moles of the formed unsaturated carboxylic acid.

The bulk specific gravity of the catalyst component particles and the specific gravity of the first-molded product were measured in the following manner.

Bulk specific gravity: Measured in accordance with a method described in JIS K6721

Specific gravity: Calculated by dividing the weight of the first-molded product comprising water (moisture) content by the volume of the first-molded product

Particle compression strength: Measured using a micro-compression testing machine (MCTM-200, manufactured by Shimadzu Corporation). The average compression strength was taken as the measured average value of 30 particles.

### Example 1

Five hundred (500) parts of ammonium paramolybdate, 6.2 parts of ammonium paratungstate, 1.4 parts of potassium nitrate, 27.5 parts of antimony trioxide and 49.5 parts of bismuth trioxide were added to 1,000 parts of purified water, and this mixture was heated with stirring (fluid A). Separately, 123.9 parts of ferric nitrate, 288.4 parts of cobalt nitrate and 35.1 parts of zinc nitrate were successively added to 1,000 parts of purified water and dissolved therein (fluid B). After an aqueous slurry was prepared by adding fluid B to fluid A, this aqueous slurry was spray-dried by means of a spray dryer equipped with a rotating disk centrifugal atomizer to form dry spherical particles having an average particle diameter of 60 μm. At this time, the revolution speed of the spray dryer atomizer was 11000 rpm, the inlet temperature 165° C. and the outlet temperature 125° C. These dry spherical particles were calcined at 300° C. for 1 hour to form calcined particles. The average particle diameter of the calcined particles was 52 μm, the average particle compression strength was 1.1×10⁻² N, and the bulk specific gravity was 0.90 kg/L.

To 500 parts of the calcined particles thus obtained was added 15 parts of methylcellulose, followed by dry blending. After 180 parts of purified water was mixed therewith, the resulting indeterminate shaped mixture was kneaded on a kneader until a clayish material was obtained. Thereafter, using a screw type extruder, the clayish material was extrusion-molded to obtain a cylindrical shape having a diameter of 45 mm and a length of 280 mm. Here, this first-molded product had a specific gravity of 1.95 kg/L. This first-molded product was then extrusion-molded using a piston type extruder having a cylinder with a diameter of 50 mm and a length of 300 mm to give a ring-shaped molded catalyst having an outer diameter of 6 mm, an inner diameter of 3 mm and a length of 5 mm. Vacuum deaeration during molding was not carried out.

Using a hot-air dryer, the resulting molded catalyst was dried at 110° C. and then calcined again at 510° C. for 3 hours under air-flow to obtain a finished calcined product. The composition of the elements, except oxygen (hereinafter the same), constituting the obtained finished calcined product was Mo₁₂Wo_{0.1}Bi_{0.9}Fe_{1.3}Sb_{0.8}Co_{4.2}Zn_{0.5}K_{0.06}.

This finished calcined product was charged into a reaction tube made of stainless steel, and a feeding gas comprising 5% of propylene, 12% of oxygen, 10% of water vapor and 73% of nitrogen (on a volume percentage basis) was passed through the catalyst layer at atmospheric pressure (pressure at the catalyst layer outlet) and a contact time of 3.6 seconds to react at a temperature of 310° C. As a result of the reaction, the propylene conversion ratio was 99.0%, the acrolein selectivity was 91.1%, and the acrylic acid selectivity 6.6%.

### Example 2

A molded catalyst was prepared in the same manner as that in Example 1, except that the first-molded product was made into a cylindrical shape having a diameter of 20 mm and a length of 280 mm, then subjected to a reaction. The reaction results were a propylene conversion ratio of 98.8%, acrolein selectivity of 90.7%, and acrylic acid selectivity of 6.3%.

### Example 3

A molded catalyst was prepared in the same manner as that in Example 1, except that the revolution speed of the spray dryer atomizer was 13500 rpm and the average particle diameter of the dried particles was 45 μm, then subjected to a reaction. At this time, the average particle diameter of the calcined particles was 41 μm, the average particle compression strength was 1.4×10⁻² N, the bulk specific gravity was 0.91 kg/L, and the specific gravity of the first-molded product was 1.98 kg/L.

The reaction results using the finished calcined product were a propylene conversion ratio of 99.0%, acrolein selectivity of 91.0%, and acrylic acid selectivity of 6.4%.

### Example 4

A molded catalyst was prepared in the same manner as that in Example 1, except that the amount of purified water in the fluid B was 600 parts, then subjected to a reaction. At this time, the average particle diameter of the dried particles was 59 μm, the average particle diameter of the calcined particles was 51 μm, the average particle compression strength was 5.4×10⁻² N, the bulk specific gravity was 1.12 kg/L, and the specific gravity of the first-molded product was 1.94 kg/L. The reaction results were a propylene conversion ratio of 98.9%, acrolein selectivity of 90.9%, and acrylic acid selectivity of 6.4%.

### Comparative Example 1

A molded catalyst was prepared in the same manner as that in Example 1, except that preparation was carried out without using a spray dryer for the drying of the aqueous slurry, the dried particles being prepared by heating the aqueous slurry while stirring to evaporate to give solid, then, the obtained solid product was dried for 6 hours at 130° C., and the resulting dried product was pulverized to prepare dried particles having an indeterminate shape. The dried particles were then subjected to a reaction. The average particle diameter of the calcined particles having an indeterminate shape was 140 μm, and the bulk specific gravity was 0.88 kg/L. The specific gravity of the first-molded product was 2.10 kg/L. The reaction results were a propylene conversion ratio of 98.6%, acrolein selectivity of 90.3%, and acrylic acid selectivity of 6.1%.

### Example 5

Five hundred (500) parts of ammonium paramolybdate, 12.4 parts of ammonium paratungstate, 23.0 parts of cesium nitrate, 24.0 parts of antimony trioxide and 33.0 parts of bismuth trioxide were added to 1,000 parts of purified water, and this mixture was heated with stirring (fluid A). Separately, 209.8 parts of ferric nitrate, 82.4 parts of nickel nitrate, 446.4 parts of cobalt nitrate, 31.3 parts of lead nitrate and 2.8 parts of 85% phosphoric acid were successively added to 1,000 parts of purified water and dissolved therein (fluid B). After an aqueous slurry was prepared by adding fluid B to fluid A, this aqueous slurry was spray-dried by means of a spray dryer equipped with a rotating disk centrifugal atomizer to form dry spherical particles having an average particle diameter of 60 μm. At this time, the revolution speed of the spray dryer atomizer was 11000 rpm, the inlet temperature 165° C. and the outlet temperature 125° C. These dry spherical particles were calcined at 300° C. for 1 hour, then further calcined at 510° C. for 3 hours to form calcined particles. The average particle diameter of the calcined particles was 54 μm, the average particle compression strength 1.3×10⁻² N, and the bulk specific gravity was 0.96 kg/L.

To 500 parts of the calcined catalyst material thus obtained was added 18 parts of methylcellulose, followed by dry blending. After 185 parts of purified water was mixed therewith, the resulting indeterminate shaped mixture was kneaded on a kneader until a clayish material was obtained. Thereafter, using a screw type extruder, the clayish material was extrusion-molded to obtain a cylindrical shape having a diameter of 45 mm and a length of 280 mm. Here, this first-molded product had a specific gravity of 1.94 kg/L. This first-molded product was then extrusion-molded using a piston type extruder having a cylinder with a diameter of 50 mm and a length of 300 mm to give a ring-shaped molded catalyst having an outer diameter of 5 mm, an inner diameter of 2 mm and a length of 5 mm. Vacuum deaeration during molding was not carried out.

Using a hot-air dryer, the resulting molded catalyst was dried at 110° C. and then calcined again at 400° C. for 3 hours under air-flow to obtain a finished calcined product. The composition of the elements, except oxygen, constituting the obtained finished calcined product was Mo₁₂Wo_{0.2}Bi_{0.6}Fe_{2.2}Sb_{0.7}Ni_{1.2}Co_{6.5}Pb_{0.4}P_{0.1}Cs_{0.5}.

This finished calcined product was charged into a reaction tube made of stainless steel, and a feeding gas comprising 5% of isobutylene, 12% of oxygen, 10% of water vapor and 73% of nitrogen (on a volume percentage basis) was passed through the catalyst layer at atmospheric pressure (pressure at the catalyst layer outlet) and a contact time of 3.6 seconds to react at a temperature of 340° C. As a result of the reaction, the isobutylene conversion ratio was 98.0%, the methacrolein selectivity was 89.9%, and the methacrylic acid selectivity was 4.0%.

### Example 6

A molded catalyst was prepared in the same manner as that in Example 5, except that the amount of purified water during kneading was 165 parts, then subjected to a reaction. At this time, the first-molded product specific gravity was 2.13 kg/L. The reaction results were an isobutylene conversion ratio of 97.8%, methacrolein selectivity of 89.8%, and methacrylic acid selectivity of 3.8%.

### Example 7

A molded catalyst was prepared in the same manner as that in Example 5, except that the preformed product was made into a cylindrical shape having a diameter of 25 mm and a length of 280 mm, then subjected to a reaction. At this time, the first-molded product specific gravity was 1.94 kg/L. The reaction results were an isobutylene conversion ratio of 97.9%, methacrolein selectivity of 89.8%, and methacrylic acid selectivity of 3.9%.

### Comparative Example 2

A molded catalyst was prepared in the same manner as that in Example 5, except that the first-molding was not carried out, so that the kneaded material having an indeterminate shape was directly molded by piston type extrusion-molding. The resulting product was then subjected to a reaction. The reaction results were an isobutylene conversion ratio of 97.5%, methacrolein selectivity of 89.6%, and methacrylic acid selectivity of 3.7%. In addition, the ring-shaped molded catalyst prepared according to this method was non-uniform and a molding yield was low.

### Comparative Example 3

A molded catalyst was prepared in the same manner as that in Example 5, except that preparation was carried out without using a spray dryer for the drying of the aqueous slurry, the dried particles being prepared by heating the aqueous slurry while stirring to evaporate to give solid, wherein the obtained solid product was dried for 6 hours at 130° C., and the resulting dried product was pulverized to prepare dried particles. The dried particles were then subjected to a reaction. The average particle diameter of the calcined particles having an indeterminate shape was 145 μm, and the bulk specific gravity was 0.87 kg/L. The first-molded product specific gravity was 2.11 kg/L. The reaction results were an isobutylene conversion ratio of 97.4%, methacrolein selectivity of 89.5%, and methacrylic acid selectivity of 3.6%.

### Example 8

Using the catalyst according to Example 5, a reaction was carried out in the same manner as in Example 5, except for changing the raw material to TBA. The reaction results were a TBA conversion ratio of 100%, methacrolein selectivity of 88.7%, and methacrylic acid selectivity of 3.1%.

### Comparative Example 4

Using the catalyst according to Comparative Example 3, a reaction was carried out in the same manner as in Comparative Example 3, except for changing the raw material to TBA. The reaction results were a TBA conversion ratio of 100%, methacrolein selectivity of 88.1%, and methacrylic acid selectivity of 2.5%.

### Industrial Applicability

The catalyst for preparing an unsaturated aldehyde and unsaturated carboxylic acid according to the present invention is excellent in catalytic activity and in selectivity of unsaturated aldehyde and unsaturated carboxylic acid, thus, the use of this catalyst allows an unsaturated aldehyde and an unsaturated carboxylic acid to be prepared at a high yield.

1-13. (canceled)

14. A process for preparing an extrusion-molded catalyst containing at least molybdenum, bismuth and iron for use in the production of an unsaturated aldehyde and an unsaturated carboxylic acid by vapor-phase catalytic oxidation of at least one compound selected from the group consisting of propylene, isobutylene, tert-butyl alcohol and methyl tert-butyl ether using molecular oxygen, said process comprising:
a step of preparing catalyst component particles as dried particles prepared by spray drying an aqueous slurry comprising molybdenum, bismuth and iron or as calcined particles prepared by further heat treating said dried particles;
a step of mixing and kneading said catalyst component particles with at least a liquid;
a first-molding step of molding the kneaded material; and
a second-molding step of molding the first-molded product to a finished shape using a piston molding machine.

15. The preparation process according to claim 14, wherein said first-molded product is in a cylindrical shape having a diameter of 0.5 times or larger and less than 1 times of the cylinder diameter of the piston molding machine used in the second-molding step.

16. The preparation process according to claim 14, wherein the specific gravity of said first-molded product is from 1.1 to 2.7 kg/L.

17. The preparation process according to claim 14, wherein average particle diameter of said catalyst component particles is from 10 to 150 μm.

18. The preparation process according to claim 14, wherein average particle compression strength of said catalyst component particles is from 9.8×10⁻⁴ to 9.8×10⁻² N.

19. The preparation process according to claim 14, wherein bulk specific gravity of said catalyst component particles is from 0.5 to 1.8 kg/L.

20. The preparation process according to claim 14, wherein vacuum deaeration is not carried out during molding of the first-molded product into a finished shape using the piston molding machine of the second-molding.

21. The preparation process according to claim 14, wherein during the first molding, a screw extruder is used for the molding.

22. The preparation process according to claim 14, wherein the amount of said liquid to be mixed with said catalyst component particles is from 35 to 55 parts by mass per 100 parts by mass of the catalyst component particles.

23. The preparation process according to claim 14, wherein said catalyst component particles are calcined particles.

24. A catalyst for the preparation of an unsaturated aldehyde and an unsaturated carboxylic acid prepared by the preparation process according to claim 14.

25. The catalyst according to claim 24, wherein the catalyst shape is a ring shape, and an outer diameter thereof is from 3 to 15 mm.

26. A preparation process of an unsaturated aldehyde and an unsaturated carboxylic acid, comprising a vapor-phase catalytic oxidation of at least one compound selected from the group consisting of propylene, isobutylene, tert-butyl alcohol and methyl tert-butyl ether using molecular oxygen using the catalyst for preparation of an unsaturated aldehyde and an unsaturated carboxylic acid according to claim 24.

27. A catalyst for the preparation of an unsaturated aldehyde and an unsaturated carboxylic acid prepared by the preparation process according to claim 15.

28. A catalyst for the preparation of an unsaturated aldehyde and an unsaturated carboxylic acid prepared by the preparation process according to claim 16.

29. A catalyst for the preparation of an unsaturated aldehyde and an unsaturated carboxylic acid prepared by the preparation process according to claim 17.

30. A catalyst for the preparation of an unsaturated aldehyde and an unsaturated carboxylic acid prepared by the preparation process according to claim 18.

31. A catalyst for the preparation of an unsaturated aldehyde and an unsaturated carboxylic acid prepared by the preparation process according to claim 19.

32. A catalyst for the preparation of an unsaturated aldehyde and an unsaturated carboxylic acid prepared by the preparation process according to claim 22.

33. A catalyst for the preparation of an unsaturated aldehyde and an unsaturated carboxylic acid prepared by the preparation process according to claim 23.
